(19) United States
(12) Patent Application Publication
(10) Pub. No.: US 2006/0063813 A1
(43) Pub. Date: Mar. 23, 2006

(54) Organic compounds

(76) Inventors: Patrick Edward Crawley, Folgensbourg (FR); Adrian A Spillmann, Willage Neuf (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/531,802

(22) PCT Filed: Oct. 16, 2003
(86) PCT No.: PCT/EP03/11498

§ 371 Date: Apr. 18, 2005

(30) Foreign Application Priority Data
Oct. 17, 2002 (GB) 0224198.2

(51) Int. Cl. A61K 31/433

(52) U.S. Cl. 514/362

(57) ABSTRACT

A pharmaceutical composition for treatment of pain, comprises in combination a benzothiadiazole derivative as defined and a COX-2 inhibitor for simultaneous, sequential or separate use. Also provided is a method of treating a patient suffering from pain, comprising administering to the patient an effective amount of a benzothiadiazole derivative as defined and an effective amount of a COX-2 inhibitor.

This invention relates to COX-2 inhibitors, in particular to combined use of COX-2 inhibitors with muscle relaxants, and compositions containing such combinations.

Accordingly the invention provides a pharmaceutical composition for treatment of pain, which comprises in combination a benzothiadiazole derivative of formula I 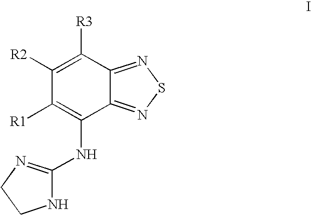
wherein each R1, R2 and R3 independently, is hydrogen, halogen, C₁-C₇ alkyl, C₁-C₇ alkoxy, nitro, cyano, hydroxy or C₁-C₇ alkylthio; and a COX-2 inhibitor for simultaneous, sequential or separate use.

Further the invention provides the use of a COX-2 inhibitor for the preparation of a medicament, for use in combination with a benzothiadiazole derivative of formula I as defined above, for treatment of pain.

In the alternative the invention provides use of a benzothiadiazole derivative of formula I as defined above, for the preparation of a medicament for use in combination with a COX-2 inhibitor for treatment of pain.

In a further aspect the invention provides a method of treating a patient suffering from pain comprising administering to the patient an effective amount of a benzothiadiazole derivative of formula I as defined above, and an effective amount of a COX-2 inhibitor.

In yet further aspects the invention provides:
(i) A package comprising a benzothiadiazole derivative of formula I as defined above, together with instructions for use in combination with a COX-2 inhibitor for treatment of pain, or
(ii) A package comprising a COX-2 inhibitor together with instructions for use in combination with a benzothiadiazole derivative of formula I as defined above, for treatment of pain.

Pain in general may be treated in accordance with the present invention including both nociceptive and inflammatory pain. In particular the combination treatment of the invention may be used for the treastment of musculoskeletal pain, especially lower back pain.

In the present description the terms “treatment” or “treat” refer to both prophylactic or preventative treatment as well as curative or disease modifying treatment, including treatment of patients at risk of suffering pain as well as patients who are already suffering pain.

In formula halogen preferably signifies bromine or chlorine.

The compounds of formula I are capable of tautomerisation and use of the tautomers thereof is included within the scope of the invention.

Preferred compounds of formula I include:

7-chloro-4-(2-imidazolin-2-yl-amino)-2,1,3,-benzothiadiazole;

4-(2-imidazolin-2-yl-amino)-7-methyl-2,1,3,-benzothiadiazole;

7-chloro-4-(2-imidazolin-2-yl-amino)-5-methyl-2,1,3,-benzothiadiazole;

5,7-dimethyl-4-(2-imidazolin-2-yl-amino)-2,1,3,-benzothiadiazole;

5-chloro-4-(2-imidazolin-2-yl-amino)-7-methyl-2,1,3,-benzothiadiazole;

5,7-dichloro-4-(2-imidazolin-2-yl-amino)-2,1,3,-benzothiadiazole;

5,6-dimethyl-4-(2-imidazolin-2-yl-amino)-2,1,3,-benzothiadiazole;

7-hydroxy-4-(2-imidazolin-2-yl-amino)-2,1,3,-benzothiadiazole;

5,6-dichloro-4-(2-imidazolin-2-yl-amino)-2,1,3,-benzothiadiazole;

6,7-dichloro-4-(2-imidazolin-2-yl-amino)-2,1,3,-benzothiadiazole;

4-(2-imidazolin-2-yl-amino)-7-methoxy-2,1,3,-benzothiadiazole;

5-bromo-7-chloro-4-(2-imidazolin-2-yl-amino)-2,1,3,-benzothiadiazole;

7-bromo-5-chloro-4-(2-imidazolin-2-yl-amino)-2,1,3,-benzothiadiazole;

4-(2-imidazolin-2-yl-amino)-2,1,3,-benzothiadiazole;

4-(2-imidazolin-2-yl-amino)-5-methyl-2,1,3,-benzothiadiazole;

4-(2-imidazolin-2-yl-amino)-5-chloro-2,1,3,-benzothiadiazole;

4-(2-imidazolin-2-yl-amino)-5-methoxy-2,1,3,-benzothiadiazole;

5-ethyl-4-(2-imidazolin-2-yl-amino)-2,1,3,-benzothiadiazole, and

5-bromo4-(2-imidazolin-2-yl-amino)-2,1,3,-benzothiadiazole.

The most preferred compound of formula I for use in the invention is 5-chloro-4-(2-imidazolin-2-yl-amino)-7-methyl-2,1,3,-benzothiadiazole, alternatively known as 5-chloro-N-(4,5-dihydro-1H-imidazol-2-yl)-2,1,3-benzothiadazol-4-amine, DS-103-282, Sirdalud and Ternelin.

Processes for the preparation of the compounds of formula I is described in the literature; for example, in U.S. Pat. No. 3,843,668.

The COX-2 inhibitors used in the pharmaceutical compositions and treatment methods of the present invention are typically those which have an IC₅₀ for COX-2 inhibition less than about 2 μM and an IC₅₀ for COX-1 inhibition greater than about 5 μM, e.g. when measured in the assays described by Brideau et al.in Inflamm. Res. 45:68-74 (1996). Preferably the COX-2 inhibitor has a selectivity ratio of at least 10, more preferably at least 40, for COX-2 inhibition over COX-1 inhibition.

Thus, for example, suitable COX-2 inhibitors for use in the invention may include the following compounds or derivatives thereof or a pharmaceutically acceptable salt thereof, or any hydrate thereof: rofecoxib, etoricoxib, celecoxib, valdecoxib, parecoxib, or a 5-alkyl-2-arylaminophenylacetic acid derivative COX-2 inhibitor, e.g. of formula V as defined below.

Alternative classes of COX-2 inhibitor compounds for use in the invention include those described in U.S. Pat. No. 6,136,804 (Merck).

COX-2 inhibitors of formula V are particularly preferred for use in the present invention.

Thus in preferred embodiments the COX-2 inhibitor for use in the present invention comprises a compound of formula V 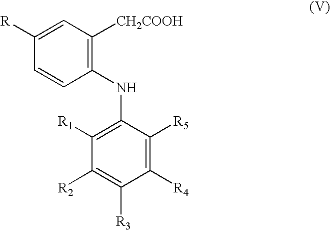
wherein R is methyl or ethyl;
R₁ is chloro or fluoro;
R₂ is hydrogen or fluoro;
R₃ is hydrogen, fluoro, chloro, methyl, ethyl, methoxy, ethoxy or hydroxy,
R₄ is hydrogen or fluoro; and
R₅ is chloro, fluoro, trifluoromethyl or methyl.

Above and elsewhere in the present description the terms “a benzothiadiazole derivative” and “COX-2 inhibitor” include, as appropriate, pharmaceutically acceptable salts and esters thereof.

Particularly preferred compounds of formula V are those wherein R is methyl or ethyl; R₁ is chloro or fluoro; R₂ is hydrogen; R₃ is hydrogen, fluoro, chloro, methyl or hydroxy, R₄ is hydrogen; and R₅ is chloro, fluoro or methyl; pharmaceutically acceptable salts thereof; and pharmaceutically acceptable esters thereof.

A particularly preferred embodiment relates to the compounds of formula V wherein R is methyl or ethyl; R₁ is fluoro; R₂ is hydrogen; R₃ is hydrogen, fluoro or hydroxy, R₄ is hydrogen; and R₅ is chloro; pharmaceutically acceptable salts thereof; and pharmaceutically acceptable prodrug esters thereof

Another particularly preferred embodiment of the invention relates to compounds of formula V wherein R is ethyl or methyl; R₁ is fluoro; R₂ is hydrogen or fluoro; R₃ is hydrogen, fluoro, ethoxy or hydroxy; R₄ is hydrogen or fluoro; and R₅ is chloro, fluoro or methyl; pharmaceutically acceptable salts thereof; and pharmaceutically acceptable prodrug esters thereof.

Further are said compounds wherein R is methyl or ethyl; R₁ is fluoro; R₂-R₄ are hydrogen or fluoro; and R₅ is chloro or fluoro; pharmaceutically acceptable salts thereof; and pharmaceutically acceptable prodrug esters thereof.

A further embodiment of the invention relates to the compounds of formula V wherein R is methyl or ethyl; R₁ is fluoro; R₂ is fluoro; R₃ is hydrogen, ethoxy or hydroxy; R₄ is fluoro; and R₅ is fluoro; pharmaceutically acceptable salts thereof; and pharmaceutically acceptable prodrug esters thereof.

Another embodiment of the invention relates to the compounds of formula V wherein R is methyl; R₁ is fluoro; R₂ is hydrogen; R₃ is hydrogen or fluoro; R₄ is hydrogen; and R₅ is chloro; pharmaceutically acceptable salts thereof; and pharmaceutically acceptable prodrug esters thereof

Particularly preferred embodiments of the invention relate to compounds of formula V

(a) wherein R is methyl; R₁ is fluoro; R₂ is hydrogen; R₃ is hydrogen; R₄ is hydrogen; and R₅ is chloro; pharmaceutically acceptable salts thereof; and pharmaceutically acceptable prodrug esters thereof;

(b) wherein R is methyl; R₁ is fluoro; R₂ is hydrogen; R₃ is fluoro; R₄ is hydrogen; and R₅ is chloro; pharmaceutically acceptable salts thereof; and pharmaceutically acceptable prodrug esters thereof;

(c) wherein R is ethyl; R₁ is fluoro; R₂ is fluoro; R₃ is hydrogen; R₄ is fluoro; and R₅ is fluoro; pharmaceutically acceptable salts thereof; and pharmaceutically acceptable prodrug esters thereof; and

(d) wherein R is ethyl; R₁ is chloro; R₂ is hydrogen; R₃ is chloro; R₄ is hydrogen; and R₅ is methyl; pharmaceutically acceptable salts thereof; and pharmaceutically acceptable prodrug esters thereof.

Most preferably the COX-2 inhibitor of formula V is 5-methyl-2-(2′-chloro-6′-fluoroanilino)phenylacetic acid, or a salt or ester thereof

Pharmaceutically acceptable prodrug esters of the compounds of formula V are ester derivatives which are convertible by solvolysis or under physiological conditions to the free carboxylic acids of formula V. Such esters are e.g. lower alkyl esters (such as the methyl or ethyl ester), carboxy-lower alkyl esters such as the carboxymethyl ester, nitrooxy-lower alkyl esters (such as the 4-nitrooxybutyl ester), and the like. Preferred prodrugs are the compounds of formula Ia

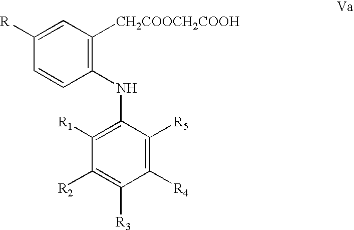

wherein R and R₁-R₅ have meaning as defined hereinabove for compounds of formula V; and pharmaceutically acceptable salts thereof.

Compounds of formula V and Va and their synthesis are described in published international patent applications Nos. WO 99/11605 and WO 01/23346, the teachings of which are incorporated herein by reference.

Pharmacologically acceptable salts of benzothiadiazole derivatives and COX-2 inhibitors are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

The Agents of the Invention, i.e. the COX-2 inhibitor and the benzothiadiazole derivative are preferably used in the form of pharmaceutical preparations that contain the relevant therapeutically effective amount of of each active ingredient (either separately or in combination) optionally together with or in admixture with inorganic or organic, solid or liquid, pharmaceutically acceptable carriers which are suitable for administration. The Agents of the Invention may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

The particular mode of administration and the dosage may be selected by the attending physician taking into account the particulars of the patient, especially age, weight, life style, activity level, and disease state as appropriate

Preferably, both the COX-2 inhibitor and benzothiadiazole derivative pharmaceutical compositions are adapted for oral or parenteral (especially oral) administration. Intravenous and oral, first and foremost oral, adminstration is considered to be of particular importance. Preferably the COX-2 inhibitor active ingredient is in oral form.

The dosage of COX-2 inhibitor administered is dependent on the species of warm-blooded animal (mammal), the body weight, age and individual condition, and on the form of administration. A unit dosage for oral administration to a mammal of about 50 to 70 kg may contain between about 5 and 1500 mg, e.g. from 100-1000 mg, preferably 200-800 mg of the active ingredient.

COX-2 inhibitor formulations in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 1 mg to about 1500 mg of the active ingredient.

COX-2 inhibitor formulations in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 1 mg to about 1500 mg of the active ingredient.

Similarly the dosage of benzothiadiazole derivative administered is dependent on the species of warm-blooded animal (mammal), the body weight, age and individual condition, and on the form of administration. In general, the daily dosage of benzothiadiazole derivative varies between about 0.01 mg/kg and about 100 mg/kg. Suitable unit dosage forms, such as dragées, tablets or suppositories, preferably contain from about 10 to about 400 mg of benzothiadiazole derivative. Dosage units for oral administration preferably contain between 10% and 90% by weight of benzothiadiazole derivative.

Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following examples are intended to illustrate the invention and are not to be construed as being limitations thereon.

### EXAMPLES

### A. Formulation Examples

### Example 1

TABLE 1

| | Amount per 200 mg |
|---|---|
| Ingredient | tablet batch (kg) |
| Core | |
| Granulation | |
| 5-methyl-2-(2′-chloro-6′- | 50** |
| fluoroanilino)phenylacetic acid drug | |
| substance | |
| Microcrystalline cellulose, NF (PH | 12.85 |
| 101) | |
| Lactose monohydrate, NF | 11.65 |
| Croscarmellose sodium, NF | 1 |
| Povidone, USP | 4 |
| Titanium dioxide, USP | 2 |
| Water, purified***, USP | 20.375 |
| Extra-granular Phase | |
| Microcrystalline cellulose, NF (PH | 13 |
| 102) | |
| Croscarmellose sodium, NF | 3 |
| Titanium dioxide, USP | 2 |
| Magnesium stearate, NF | 0.5 |
| Coating | |
| Opadry white | 2.801**** |
| Opadry yellow | 2.0**** |
| Opadry red | 0.4**** |
| Opadry black | 0.0504**** |
| Water, purified***, USP | 29.758**** |

**The weight of drug substance is taken with reference to the dried substance (100 percent) on the basis of the assay value (factorization). The difference in weight is adjusted by the amount of microcrystalline cellulose used.
***Removed during processing.
****Includes a 50% excess for loss during the coating process.

Table 1, above, sets out the formula for a batch of approximately 250,000 immediate release film-coated tablets of 5-methyl-2-(2′-chloro-6′-fluoroanilino)-phenylacetic acid. To make the tablets, titanium dioxide is dispersed in water, followed by the addition of povidone and mixing for 20 minutes to make a povidone/titanium dioxide suspension. The drug substance, lactose, microcrystalline cellulose, and croscarmellose are mixed in a high shear mixer (e.g., a Collette Gral) for 5 minutes to form a drug mixture. The drug mixture is granulated in the high shear mixer with the povidone/titanium dioxide suspension. The suspension is pumped at a rate of 3 kg/min into the drug mixture. The resulting mixture is mixed an additional 90 seconds after all the suspension is added. The wet granulation is dried in a fluid bed dryer, using an inlet air temperature of 50° C. The residual water target is 3.5% (with a permissible range of 2.5-4.5%). The dried granulation is passed through a screen using a mill (oscillator) and a 30 mesh screen. The previous steps are repeated to make a second granulation.

The extra-granular phase titanium dioxide is passed through a 60 mesh hand screen. The dry granulations are mixed with the extra-granular phase microcrystalline cellulose, croscarmellose sodium and titanium dioxide in a twin shell mixer for 300 revolutions to form a penultimate mixture. Magnesium stearate is passed through a 60 mesh hand screen and is mixed with the penultimate mixture in a twin shell mixer for 50 revolutions to form a tableting mixture. The tableting mixture is pressed into tablets using a tablet press and oval punches.

The coating powders (Opadry) are mixed with purified water to make a 15% w/w coating suspension. The tablets are film coated with the coating suspension in a coating pan using 60° C. to 75° C. inlet air temperature.

Table 2 sets out the contents of a 200 mg 5-methyl-2-(2′-chloro-6′-fluoroanilino)phenylacetic acid film-coated tablet.

TABLE 2

| | Theoretical | |
|---|---|---|
| Ingredient | amount [mg] | Function |
| Core | | |
| 5-methyl-2-(2′-chloro-6′- | 200 | Active |
| fluoroanilino)phenylacetic | | substance |
| acid drug substance | | |
| Microcrystalline cellulose (PH | 51.4 | Filler |
| 101) | | |
| Lactose | 46.6 | Filler |
| Povidone | 16 | Binder |
| Titanium dioxide | 8 | Color |
| Croscarmellose sodium | 4 | Disintegrant |
| Water, purified* | Q.S. | Granulating |
| | | liquid |
| Extragranular phase | | |
| Microcrystalline cellulose (PH | 52 | Filler |
| 102) | | |
| Croscarmellose sodium | 12 | Disintegrant |
| Titanium dioxide | 8 | Color |
| Magnesium stearate | 2 | Lubricant |
| Core weight | 400 | |
| Coating | | |
| Opadry white (00F18296) | 7.4676 | Color |
| Opadry yellow (00F12951) | 5.3312 | Color |
| Opadry red (00F15613) | 1.0668 | Color |
| Opadry black (00F17713) | 0.1344 | Color |
| Water, purified* | Q.S. | Coating solvent |
| Total weight | 414 | |

*removed during processing

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1

### Example 2

An alternative formulation is as set out in Table 3, with information about as percentage w/w, mg/dose, and kg/50,000 tablet batch.

TABLE 3
Alternative formulation composition

| % w/w | Ingredient | Mg/dose | Kg/batch |
|---|---|---|---|
| | Granulation | | |
| 65.04 | 5-methyl-2-(2′-chloro-6′-fluoroanilino) | 400.00 | 20.00 |
| | phenylacetic acid drug substance | | |
| 2.15 | Croscarmellose sodium, NF (Ac-Di-Sol) | 13.22 | 0.661 |
| 6.60 | Povidone K30, USP | 40.59 | 2.029 |
| 18.12 | Purified water, USP* | Qs | Qs |
| | Blending | | |
| 23.56 | Microcrystalline Cellulose, NF (Avicel | 144.90 | 6.066 |
| | PH 102) | | |
| 2.15 | Croscarmellose sodium, NF (Ac-Di-Sol) | 13.22 | 0.553 |
| 0.50 | Magnesium Stearate, NF (vegetable | 3.07 | 0.128 |
| | source) | | |
| | Film Coating | | |
| 84.46 | Opadry, Global White 00F18296 | 15.2028 | 0.296637 |
| 14.03 | Opadry, Global Red 00F15613 | 2.5254 | 0.049275 |
| 1.51 | Opadry, Global Black 00F17713 | 0.2718 | 0.005303 |
| | Purified Water, USP* | Qs | 1.990218 |
| | Film Coated Tablet Weight | 633.00 | |

*Does not appear in final product. Percentage of water added used for granulation based on the dry weight of drug substance and croscarmellose sodium.

The batch is granulated as described in Example 1. The granulation is dried to residual moisture (% LOD) of 1.79%. The formulation process is the same as for the development batches as described above, except for the additional step of coating with Opadry in a coating pan. The coating powders (Opadry) are mixed with purified water to make a 15% w/w coating suspension. The tablets are film coated with the coating suspension in a coating pan using 60° C. to 75° C. inlet air temperature. Based on friability data, a target force of 18 KN (16-20 KN range) is used to compress the remainder of the batch, resulting in acceptable friability (less than 0.5%) and the disintegration times of less than 5 mins. The ejection force is approximately 800 N throughout the compression run. This demonstrates that the blend is lubricated adequately. No picking/sticking is observed on the punch surfaces after 225 minutes. Thus, a smaller size tablet with high drug loading (65%) is achieved using a high shear granulation process, using 17×6.7 mm ovaloid tooling to get tablets with acceptable hardness and friability characteristics.

In addition, the tablet formulations may contain 5-methyl-2-(2′-chloro-6′-fluoroanilino)benzyl alcohol and/or 5-methyl-2-(2′-chloro-6′-fluoroanilino)benzoic acid in an amount between about 0.01 and 2% by weight, more specifically between about 0.1 and 1%.

### Example 3

Wet Granulated Tablet Composition

| Amount per tablet | | Ingredient |
|---|---|---|
| 25 | mg | COX-2 inhibitor |
| 79.7 | mg | Microcrystalline cellulose |
| 79.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

Tablet dose strengths of between 5 and 125 mg can be accomodated by varying total weight, and the ratio of the first three ingredients. Generally it is preferable to maintain a 1:1 ratio for microcrystalline cellulose: lactose monohydrate.

### Example 4

Hard Gelatine Capsule Composition

| Amount per capsule | | Ingredient |
|---|---|---|
| 25 | mg | COX-2 inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accomodated by varying total fill weight, and the ratio of the first three ingredients. Generally it is preferable to maintain a 1:1 ratio for microcrystalline cellulose:lactose monohydrate.

### Example 5

Oral Solution

| Amount per 5 mL | | Ingredient |
|---|---|---|
| 50 | mg | COX-2 inhibitor |
| to 5 | mL with | Polyethylene oxide 400 |

### Example 6

Intravenous Infusion

| Amount per 200 mL dose | | Ingredient |
|---|---|---|
| 1 | mg | COX-2 inhibitor |
| 0.2 | mg | Polyethylene oxide 400 |
| 1.8 | mg | Sodium chloride |
| to 200 | mL | Purified water |

### Example 7

Benzothiadiazole Derivative Formulations

An example of a tablet composition comprises 40 mg of 5-chloro-4-(2-imidazolin-2-yl-amino)-2,1,3-benzothiazole, 70 mg of lactose, 5 mg of maize starch, 5 mg of talc and 0.1 mg of magnesium stearate.

### Example 8

### Treatment of Patients

Assumptions:

| | | |
|---|---|---|
| 1) | Two formulations: | 200 mg Prexige plus 300 mg Sirdalud |
| | | 200 mg Prexige plus 600 mg Sirdalud |
| 2) | b.i.d. dosing | |
| 3) | limited titration | |

| | |
|---|---|
| 4) | effective dose Sirdalud = 900-1200 mg/day |
| | effective dose Prexige = 400 mg/day |
| 5) | sample size would have to estimated by a statistician |
| 6) | trial timeline is set up to achieve POC but possibly not statistical |
| | significance |

Design: double-blind, placebo-controlled, parallel group, multicenter

Duration: 4 to 6 weeks including screening

Patient population:
inclusion criteria—male or female ≧to 18 years old
   low back pain (below T6 and above gluteal fold) that may radiate to leg
   pain lasting more than three months
   pain present on five out of seven days
   VAS score ≧to 40 mm on four of the last seven days
   comprehensive history and physical examination including focused neurological examination
exclusion criteria—unstable spinal segment
   progressive neurological deficits
   excluded drugs: all other NSAIDs, opioids, TCAs, AEDs, oral steroids except for treatment of asthma or skin conditions, steroid injections
   other pain conditions that may interfere with assessment of the low back pain
   patients previously treated with either Prexige or Sirdalud
   patients with hypersensitivity to carbamazepine, oxcarabazepine or lumiracoxib and other non-steroidal anti-inflammatories including aspirin
   patients with active disability compensation claims or any litigation related to their radiculopathic pain.

Variables:
primary efficacy variable—VAS
secondary efficacy variables—responder rate, sleep assessment, SF-36, POMS, assessment of back mobility and low-back pain specific QOL

Suggested visit schedule:

| | |
|---|---|
| visit 1 (day − 14 to day − 1) | screening |
| visit 2 (day 1) | randomization, titration and treatment |
| visit 3 (day 21) | withdrawal |
| visit 4 (day 28) | final visit |

The withdrawal phase can be eliminated to give 4 weeks total treatment (I week titration, 3 weeks maintenance).

Titration and maintenance dosing schedule:

| Day | AM Dose^{a} | PM Dose^{a} | Total Daily Doses^{a} |
|---|---|---|---|
| 1 | 0 | 200/300 | 200/300 |
| 2 | 200/300 | 200/300 | 400/600 |
| 3 | 200/300 | 200/300 | 400/600 |
| 4 | 200/300 | 200/600 | 400/900 |
| 5 | 200/300 | 200/600 | 400/900 |
| 6 | 200/600 | 200/600 | 400/1200 |
| 7-21 | 200/600 | 200/600 | 400/1200 |
| 22-28 | 0 | 0 | 0 |

^{a}expressed as mg Prexige/mg Sirdalud

1. A pharmaceutical composition for treatment of pain, which comprises in combination a benzothiadiazole derivative of formula I 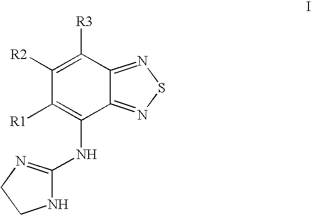
wherein each R1, R2 and R3 independently, is hydrogen, halogen, C₁-C₇ alkyl, C₁-C₇ alkoxy, nitro, cyano, hydroxy or C₁-C₇ alkylthio; and a COX-2 inhibitor for simultaneous, sequential or separate use.

2. (canceled)

3. (canceled)

4. A method of treating a patient suffering from pain comprising administering to the patient an effective amount of a benzothiadiazole derivative of formula I, 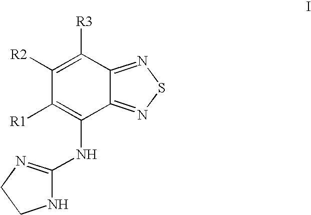
wherein each R1, R2 and R3 independently, is hydrogen, halogen, C₁-C₇ alkyl, C₁-C₇ alkoxy, nitro, cyano, hydroxy or C₁-C₁ alkylthio, and an effective amount of a COX-2 inhibitor.

5. A package comprising a benzothiadiazole derivative of formula I, 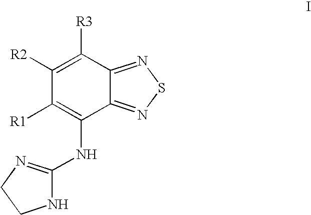
wherein each R1, R2 and R3 independently, is hydrogen, halogen, C₁-C₇ alkyl, C₁-C₇ alkoxy, nitro, cyano, hydroxy or C₁-C₇ alkyithio, for treatment of pain according to a method wherein said benzothiadiazole derivative of formula I is used in combination with a COX-2 inhibitor for treatment of pain, or a package comprising a COX-2 inhibitor for treatment of pain according to a method wherein said COX-2 inhibitor is used combination with a benzothiadiazole derivative of formula I as defined above.

6. A composition according to claim 1 in which the COX-2 inhibitor is selected from the group consisting of rofecoxib, etoricoxib, celecoxib, valdecoxib, parecoxib, and a 5-alkyl-2-arylaminophenylacetic acid derivative COX-2 inhibitor, or a pharmaceutically acceptable salt, or any hydrate thereof.

7. A composition according to claim 1 in which the COX-2 inhibitor is a compound of formula V 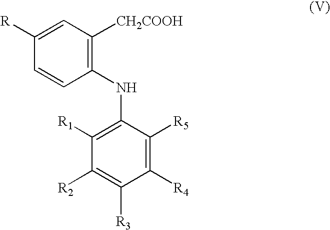
wherein R is methyl or ethyl;
R₁ is chloro or fluoro;
R₂ is hydrogen or fluoro;
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl, or a pharmaceutically acceptable salt or ester thereof.

8. A composition according to claim 7 in which the COX-2 inhibitor is 5-methyl-2-(2′-chloro-6′-fluoroanilino)phenylacetic acid, or a pharmaceutically acceptable salt or ester thereof.

9. A composition according to claim 1 in which the benzothiadiazole derivative is 5-chloro4-(2-imidazol-2-ylamino)-2,1,3-benzothiadiazole.
